(19) United States
(12) Patent Application Publication
(10) Pub. No.: US 2025/0252562 A1
(43) Pub. Date: Aug. 7, 2025

(54) METHOD FOR ESTIMATING HEART RATE ON BASIS OF CORRECTED IMAGE, AND DEVICE THEREFOR

(71) Applicants: Youn Jun KIM, Seoul (KR); BIOCONNECT INC., Seoul (KR)

(72) Inventors: Youn Jun KIM, Seoul (KR); Young Su JEON, Gwacheon-si, Gyeonggi-do (KR)

(21) Appl. No.: 18/856,642

(22) PCT Filed: Apr. 13, 2023
(86) PCT No.: PCT/KR2023/005047

§ 371 Date: Oct. 14, 2024

(30) Foreign Application Priority Data
Apr. 14, 2022 (KR) 10-2022-0046423

(51) Int. Cl. G06T 7/00; A61B 5/024; G06T 5/50; G06T 5/60; G06T 5/94; G06T 7/11

(52) U.S. Cl. CPC G06T 7/0012; A61B 5/02416; G06T 5/50; G06T 5/60; G06T 5/94; G06T 7/11; G06T 2207/10024; G06T 2207/20081; G06T 2207/30076; G06T 2207/30104; G06T 2207/30201

(57) ABSTRACT

Disclosed in one embodiment of the present invention is a method for estimating a heart rate on the basis of a corrected image, the method comprising the steps of: acquiring a serial image; extracting a region of interest (ROI) from the acquired serial image; converting, from an RGB space to a YCbCr space, the color space of the extracted ROI; calculating a weighted value applied to the serial image, by inputting the converted result to a learning model; applying the calculated weighed value to the serial image and generating a serial image with the ROI corrected; and estimating the heart rate of a person included in the corrected serial image, by analyzing the ROI of the corrected serial image.

### TECHNICAL FIELD

The present invention relates to a method for estimating a heart rate based on a corrected image and a device therefor, and more specifically, to a method for estimating a heart rate of a remote photoplethysmography (rPPG) method using an image with an improved low-illumination effect, and a device for implementing the method.

### BACKGROUND ART

In accordance with the development of image technology, as a high frame rate image may be obtained, a method for estimating a heart rate of a real person included in an image through a remote photoplethysmography (PPG) method has been disclosed and variously used.

However, when the image is captured in a low-illumination environment or a low-illumination region is detected only in a part of the image, there is a problem in that it is difficult to extract a heart rate of a person in the image using the rPPG method, and a robust model for detecting a minute change in the tone of blood vessels in the skin of the person in the image is required.

As one method for increasing illumination for a partial region of an image, there is a method for increasing a contrast ratio of the image in order to improve visibility of a dark region, but there is a limitation in that color distortion and excessive saturation improvement cause deterioration of a color signal, and as a result, it may not be used to detect a heart rate of a person by analyzing the image through the rPPG.

### DISCLOSURE

### Technical Problem

The technical problem to be solved by the present invention is to provide a method for estimating a heart rate using an improved image by improving a low-illumination effect of a low-illumination environment image through a learning model, and a device for implementing the method.

### Technical Solution

To solve the technical problem, a method according to one embodiment of the present invention includes: acquiring a serial image; extracting a region of interest (ROI) from the acquired serial image; converting a color space of the extracted region of interest from an RGB region to a YCbCr region; calculating a weighted value applied to the serial image by inputting a conversion result into a learning model; generating a serial image in which the region of interest is corrected by applying the calculated weighted value to the serial image; and estimating a heart rate of a person included in the corrected serial image by analyzing the region of interest of the corrected serial image. To solve the technical problem, a device according to one embodiment of the present invention includes: a serial image acquisition unit that acquires a serial image; a region of interest (ROI) extraction unit that extracts a region of interest from the acquired serial image; a color space conversion unit that converts a color space of the extracted region of interest from an RGB region to a YCbCr region; a weighted value calculation unit that calculates a weighted value applied to the serial image by inputting a conversion result into a learning model; a corrected image generation unit that generates a serial image in which the region of interest is corrected by applying the calculated weighted value to the serial image; and a heart rate estimation unit that estimates a heart rate of a person included in the corrected serial image by analyzing the region of interest of the corrected serial image.

One embodiment of the present invention may provide a computer-readable recording medium that stores a program for executing the method.

### Advantageous Effects

According to the present invention, a low-illumination effect may be improved by amplifying illumination of a low-illumination environment image.

In addition, according to the present invention, a more accurate heart rate waveform may be obtained by estimating a heart rate of the rPPG method using an image with an improved low-illumination effect.

### DESCRIPTION OF DRAWINGS

FIG. 1 is a block diagram showing one example of a heart rate estimation device according to the present invention.

FIG. 2 is a block diagram showing a sub-modules included in a processing unit described in FIG. 1.

FIG. 3 is a schematic diagram showing a step of calculating a weighted value applied to a serial image by inputting a conversion result into a learning model and a step of generating a serial image in which a region of interest is corrected by applying the calculated weighted value to the serial image, according to one embodiment of the present invention.

FIG. 4 is a flowchart showing one example of a method for calculating a weighted value from a low-illumination environment image through the learning model, according to one embodiment of the present invention.

FIG. 5 is a flowchart showing one example of a method for calculating a weighted value from a general environment image through the learning model, according to one embodiment of the present invention.

FIG. 6 is a flowchart showing one example of a method for generating a corrected serial image using the calculated weighted value according to FIGS. 5 and 6, according to one embodiment of the present invention.

FIG. 7 is a flowchart showing one example of a method for estimating a heart rate based on the corrected image, according to one embodiment of the present invention.

### BEST MODE

To solve the technical problem, a method according to one embodiment of the present invention includes: acquiring a serial image; extracting a region of interest (ROI) from the acquired serial image; converting a color space of the extracted region of interest from an RGB region to a YCbCr region; calculating a weighted value applied to the serial image by inputting a conversion result into a learning model; generating a serial image in which the region of interest is corrected by applying the calculated weighted value to the serial image; and estimating a heart rate of a person included in the corrected serial image by analyzing the region of interest of the corrected serial image.

In the method, the acquiring of the serial image may include acquiring a serial image of 30 fps or greater using an RGB camera, the serial image may include a facial region of the person, and the region of interest (ROI) may be the facial region of the person.

In the method, the serial image may include a low-illumination environment image and a general environment image, and in the calculating of the weighted value, the weighted value may be calculated by dividing the conversion result into a chrominance component and a luminance component, inputting the luminance component into a Retinex-model, and decomposing each of the low-illumination environment image and the general environment image into an illumination component and a reflectance component to perform learning.

In the method, the generating of the corrected image may include an adjustment step of increasing the illumination component and reducing noise of the reflectance component by applying the calculated weighted value to the low-illumination environment image, and a step of recomposing adjusted illumination component and reflectance component with the chrominance component.

To solve the technical problem, a device according to one embodiment of the present invention includes: a serial image acquisition unit that acquires a serial image; a region of interest (ROI) extraction unit that extracts a region of interest from the acquired serial image; a color space conversion unit that converts a color space of the extracted region of interest from an RGB region to a YCbCr region; a weighted value calculation unit that calculates a weighted value applied to the serial image by inputting a conversion result into a learning model; a corrected image generation unit that generates a serial image with the region of interest that is corrected by applying the calculated weighted value to the serial image; and a heart rate estimation unit that estimates a heart rate of a person included in the corrected serial image by analyzing the region of interest of the corrected serial image.

One embodiment of the present invention may provide a computer-readable recording medium that stores a program for executing the method.

### MODE FOR INVENTION

The present invention may apply various changes and may have various embodiments, and thus specific embodiments will be illustrated in the drawings and described in detail in the detailed description. The effects and features of the present invention and a method for achieving the effects and features will become more apparent from the embodiments which will be described in detail in conjunction with the accompanying drawings. However, the present invention is not limited to the embodiments disclosed below and may be implemented in various forms.

Hereinafter, the embodiments of the present invention will be described in detail with reference to the accompanying drawings, and when described with reference to the drawings, the same or corresponding components are given the same reference numerals, and redundant descriptions thereof will be omitted.

In the following embodiments, the terms such as “first” and “second” are used for the purpose of distinguishing one component from another component, not in a limiting sense.

In the following embodiments, the singular expression also includes the plural meaning as long as it does not differently mean in the context.

In the following embodiments, the terms such as “include” or “have” mean that there are features or components described in the specification, and do not preclude the possibility that one or more other features or components are added in advance.

When a certain embodiment may be implemented differently, a specific process order may be performed differently from the described order. For example, two processes that are continuously described may be performed substantially and simultaneously, or may be performed in an order opposite to the described order.

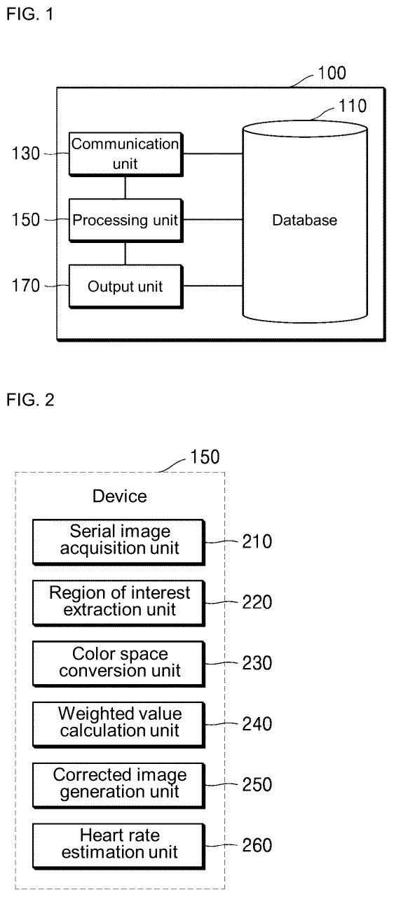

FIG. 1 is a block diagram showing one example of a heart rate estimation device according to one embodiment of the present invention.

Referring to FIG. 1, a heart rate estimation device 100 according to the present invention may include a database 110, a communication unit 130, a processing unit 150, and an output unit 170.

The heart rate estimation device 100 according to one embodiment of the present invention may correspond to at least one processor or may include at least one processor. Accordingly, the heart rate estimation device 100 and the communication unit 130, the processing unit 150, and the output unit 170 included in the heart rate estimation device 100 may be driven in the form included in a hardware device such as a microprocessor or a general-purpose computer system.

The name of each module included in the heart rate estimation device 100 shown in FIG. 1 is arbitrarily named to intuitively describe a representative function performed by each module, and when the heart rate estimation device 100 is actually implemented, a name different from the name described in FIG. 1 may be given to each module.

In addition, the number of modules included in the heart rate estimation device 100 of FIG. 1 may vary every time according to the embodiment. More specifically, the heart rate estimation device 100 of FIG. 1 includes a total of four modules, but according to the embodiment, at least two or more modules may be integrated into one module, or at least one module may be implemented in the form separated into two or more modules.

The database 110 stores various data required for the heart rate estimation device 100 to operate. For example, the database 110 may store an integrated management program for controlling an operation of the heart rate estimation device 100, and the database 110 may receive and store an analysis image received by the communication unit 130 from an external device.

The communication unit 130 performs a function of transmitting a result processed by the processing unit 150 to the outside or receiving the analysis image for determination by the processing unit 150, while communicating with the external device. The communication unit 130 may include a module for accessing a communication network and performing authentication in order to use various wired/wireless communication networks such as a data network, a mobile communication network, and the Internet.

The processing unit 150 processes data received by the communication unit 130 and data to be transmitted by the communication unit 130. More specifically, the processing unit 150 estimates a heart rate of a person included in an image by analyzing the image.

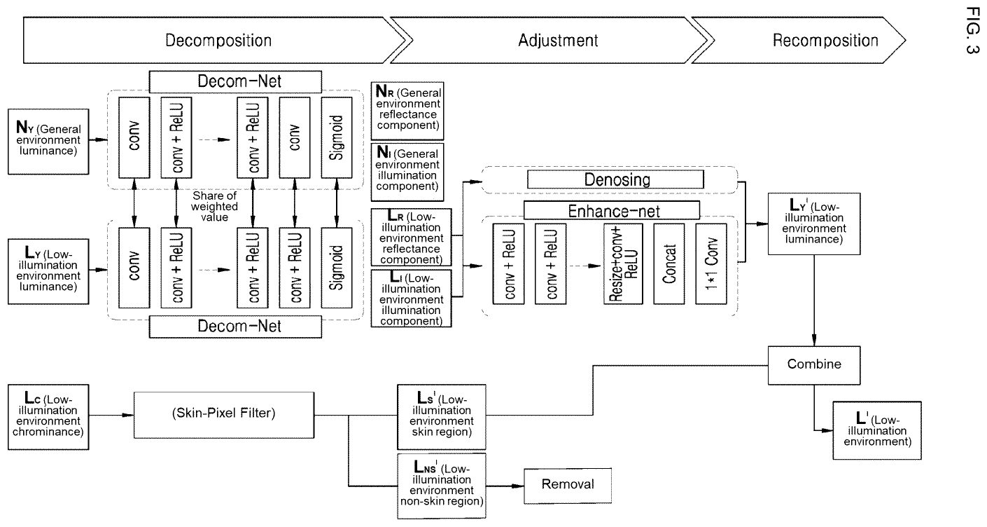

The processing unit 150 may include at least two or more sub-modules according to functions to be performed, and a detailed operation of the processing unit 150 will be described later with reference to FIG. 2.

The output unit 170 performs a function of calculating and outputting various data by receiving a command of the processing unit 150. For example, the output unit 170 may output result data processed by the processing unit 150 to transmit the result data to the communication unit 130.

FIG. 2 is a block diagram showing a sub-modules included in the processing unit described in FIG. 1.

Referring to FIG. 2, the processing unit 150 may include a serial image acquisition unit 210, a region of interest extraction unit 220, a color space conversion unit 230, a weighted value calculation unit 240, a corrected image generation unit 250, and a heart rate estimation unit 260. The processing unit 150 of FIG. 2 includes a total of six modules, but according to the embodiment, at least two or more modules included in the processing unit 150 may be integrated into one module, or at least one module may be implemented in the form separated into two or more modules.

In addition, the serial image acquisition unit 210, the region of interest extraction unit 220, the color space conversion unit 230, the weighted value calculation unit 240, the serial image generation unit 250, and the heart rate estimation unit 260, which are included in the processing unit 150 of the heart rate estimation device 100 of FIG. 2, may correspond to at least one processor or may include at least one processor. Accordingly, the heart rate estimation device 100 may be driven in the form included in another hardware device such as a microprocessor or a general-purpose computer system.

The serial image acquisition unit 210 performs a function of acquiring an image which is previously stored in the database 110 or received from the external device to the communication unit 130. In this case, the image refers to a serial image formed of a plurality of frames.

The serial image may include a body part of the person, and the body part included in the serial image may be a part in which the skin is exposed to enable heart rate analysis when the image is separated for each frame. For example, the serial image may include a face or arm of the person.

For example, the serial image is a result captured using an RGB camera, and a frame rate of the serial image may be 30 frames per second (fps) or greater. When the serial image is captured in a place with insufficient lighting, such as a low-illumination environment, the serial image is darkened as a whole, and only a portion of a capturing target included in the serial image may exhibit low illumination due to backlight or shading.

The region of interest extraction unit 220 may set and extract a region of interest (ROI) from the serial image acquired by the serial image acquisition unit 210.

For example, since continuous tracking of a facial region in the serial image is required for heart rate estimation, the region of interest may be a facial region of the person.

In addition, for another example, a region detection and tracking algorithm may be used to extract the region of interest, a technique such as YOLO, Fast-RCNN, SSD, or the like may be used to detect a region, and an object tracking algorithm such as Mean-shift, CAMshift, or the like may be used to track a moved position in comparison with a previous frame.

The color space conversion unit 230 may convert a color space of the region of interest extracted by the region of interest extraction unit 220.

For example, the color space conversion unit 230 may convert the color space of the region of interest from an RGB region to a YCbCr region in order to minimize a color error in the RGB region. In processing an image or a video, three elements of the RGB region have visually uniform information, whereas the YCbCr region may be expressed separately as a luminance component (Y component) and chrominance components (Cb and Cr component), thereby enabling independent management.

To this end, the color space conversion unit 230 may convert the color space of the region of interest from the RGB region to the YCbCr region through the following Equations 1 to 3.

$$Y ≡ 0.299 R + 0.587 G + 0.114 B \qquad [ Equation 1 ]$$

$${C}_{b} = - 0.16874 R - 0.33126 G + 0.5 B \qquad [ Equation 2 ]$$

$${C}_{r} = 0.5 R - 0.41869 G - 0.08131 B \qquad [ Equation 3 ]$$

In this case, R, G, B refer to color values of a RGB space, Y is a luminance component, and Cb and Cr are chrominance components.

The functions of the weighted value calculation unit 240 and the corrected image generation unit 250 will be described later with reference to FIGS. 3 to 6.

The heart rate estimation unit 260 may analyze a region of interest of the corrected serial image generated by the corrected image generation unit 250 to estimate a heart rate of the person included in the corrected serial image.

For example, the heart rate estimation unit 260 may estimate a heart rate of a person using remote photoplethysmography (rPPG), and according to the present invention, since the heart rate may be estimated by using an image in which with an improved low-illumination effect by amplifying the illumination, it is possible to accurately estimate a heart rate in various environments.

For example, the heart rate estimation unit 260 estimates the heart rate through the following steps. The heart rate estimation unit 260 obtains an average value of a chrominance signal for each frame from a plurality of images. Next, the heart rate estimation unit 260 may convert a chrominance signal for each frame, of which an average value is obtained, from a time region to a frequency region in order to remove noise from a component unrelated to the heart rate. In this case, a scheme of a Fourier transform or the like may be used as transform to the frequency domain.

Next, the heart rate estimation unit 260 treats the frequency unrelated to the heart rate as noise and removes the noise, and since a range generally recognized as the heart rate is 42 to 180 beats per minute (bpm) and the range corresponds to a frequency band of 0.7 to 3.0 Hz, a frequency region out of the range may be treated as the noise. When the noise of the chrominance signal is removed, a band pass filter (BPF) or the like may be utilized.

Finally, the heart rate estimation unit 260 may obtain a time-series heart rate waveform from which the noise is removed through an inverse transformation process. For another example, a method for estimating a heart rate by the heart rate estimation unit 260 through a remote photoplethysmography method is based on a conventional method, and Korean Registered Patent No. 10-2225557 discloses a technology of obtaining a heart rate by extracting a remote photoplethysmogram signal from a facial image.

FIG. 3 is a schematic diagram showing a step of calculating a weighted value applied to a serial image by inputting a conversion result into a learning model and a step of generating a serial image in which a region of interest is corrected by applying the calculated weighted value to the serial image, according to one embodiment of the present invention.

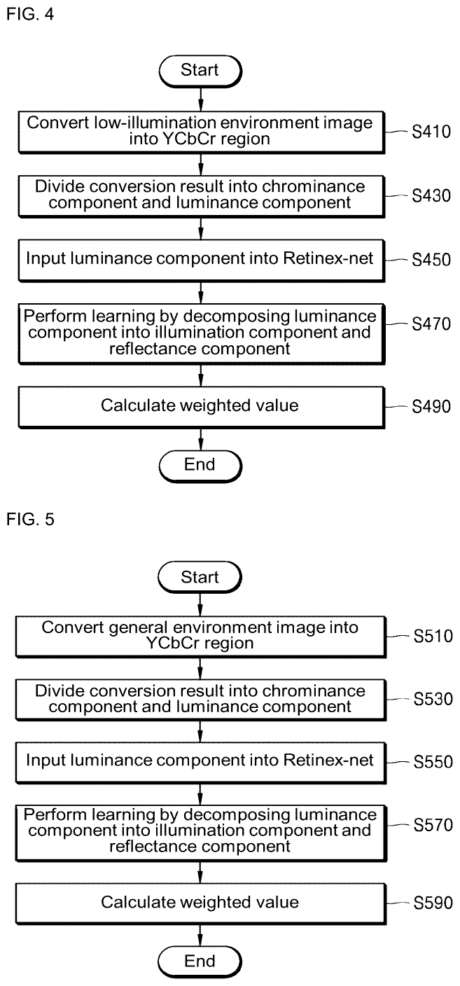

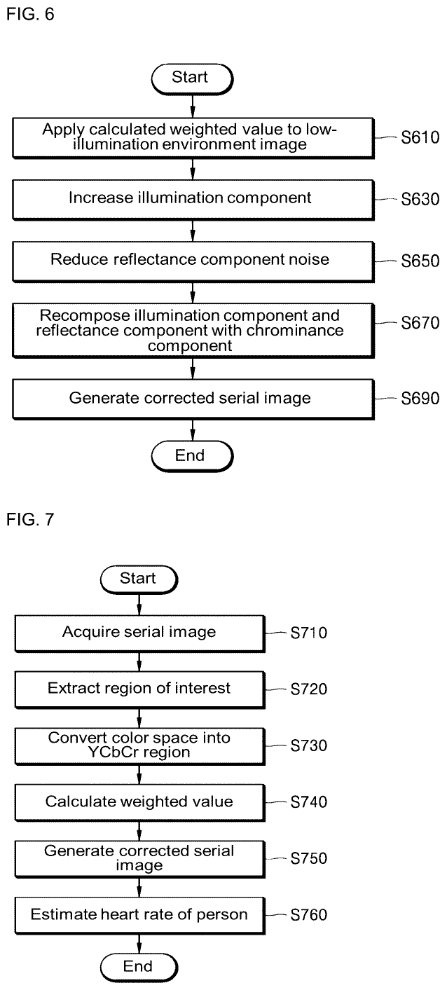

A detailed description of the step of calculating the weighted value will be described later with reference to FIGS. 4 and 5, and a detailed description of the step of generating the serial image in which the region of interest is corrected will be described later with reference to FIG. 6.

FIG. 4 and FIG. 5 are flowcharts showing one example of a method for calculating a weighted value from images of a low-illumination environment and general environment through a learning model, according to one embodiment of the present invention.

The weighted value calculation unit 240 may calculate a weighted value applied to the serial image by inputting a result converted by the color space conversion unit 230 into the learning model.

For example, the weighted value calculation unit 240 converts the color space of the region of interest from the RGB region to the YCbCr region by the color space conversion unit 230 (S410 and S510).

The weighted value calculation unit 240 may divide the conversion result into chrominance components (Cb and Cr) and a luminance component (Y) (S430 and S530).

The weighted value calculation unit 240 may input the luminance component (Y) into the learning model as an input value that is input into the learning model. For example, the learning model may be a Retinex-net decomposition for low-light enhancement model based on cognitive modeling.

The weighted value calculation unit 240 may perform learning through the learning model by decomposing the luminance component (Y) of the low-illumination environment image and the general environment image into an illumination component and a reflectance component (S470 and S570). Serial images of the low-illumination environment and the general environment share the same reflectance component, and an illumination map needs to be smooth, but needs to maintain the main structure obtained through a structure-aware total variation loss. In this case, the low-illumination environment image is an image acquired by the serial image acquisition unit 210, and may be an image captured in the low-illumination environment and having a low illumination as a whole, and the general environment image is an image having a preset value of illumination, and may be understood as comparison target data for calculating the weighted value by comparing with the low-illumination environment image.

The weighted value calculation unit 240 may finally calculate the weighted value in the low-illumination environment and the general environment (S490 and S590). The weighted values, which are trained by decomposing each of the serial images of the low-illumination environment and the general environment into the reflectance component and the illumination component, are shared with each other.

In one optional embodiment, the weighted value calculation unit 240 may compare the calculated weighted value with the preset value, and when a deviation between the calculated weighted value and the preset value exceeds a predetermined value, the process of calculating the weighted value may be extended and processed as follows.

When it is detected that the deviation between a first weighted value primarily, which is calculated by the weighted value calculation unit 240, and the preset value exceeds the predetermined value, the weighted value calculation unit 240 may calculate a second weighted value by adding a correction value to the first weighted value. In this case, the second weighted value calculated by the weighted value calculation unit 240 may be a weighted value to be finally calculated by the weighted value calculation unit 240, and the correction value added to the first weighted value may depend on the predetermined value. The predetermined value may be set to several values step by step rather than one. The reason for secondarily processing the weighted value according to the present embodiment is that when the weighted value is calculated as a too large value because the low-illumination environment image is an image captured at substantially too low illumination, an error may be included in a process of constructing the luminance component by recomposing the reflectance component with the illumination component to which the weighted value is applied in a process to be described later. When the heart rate is estimated by the remote photoplethysmography method for the image in which the illumination is excessively corrected, a low accuracy result may be calculated, and thus the weighted value calculation unit 240 may additionally correct the previously calculated weighted value as described above.

FIG. 6 is a flowchart showing one example of a method for generating a corrected serial image using the calculated weighted value according to FIGS. 4 and 5, according to one embodiment of the present invention.

The corrected image generation unit 250 may generate a serial image in which the region of interest is corrected (corrected serial image) by applying the weighted value calculated by the weighted value calculation unit 240 to the serial image.

For example, the corrected image generation unit 250 may apply the calculated weighted value to a low-illumination environment serial image (S610).

The corrected image generation unit 250 may amplify the illumination component of the low-illumination environment serial image (S630). For example, an encoder-decoder-based Enhance-net may increase the illumination component.

The corrected image generation unit 250 may reduce noise of the reflectance component of the low-illumination environment serial image (S650). For example, the reflectance component may be removed through a denoising operation. Through the adjustment step, the illumination component of the low-illumination environment serial image is increased, and the noise of the reflectance component is reduced.

The corrected image generation unit 250 may recompose the adjusted illumination component and the reflectance component with the chrominance components (Cb and Cr) (S670). Referring to FIG. 3, in step S670, the corrected image generation unit 250 may configure the corrected luminance component by recomposing the illumination component with the reflectance component.

The corrected image generation unit 250 may generate a corrected serial image with an improved low-illumination effect through the recomposition step. The corrected image generation unit 250 generates a corrected serial image by combining the corrected luminance component and the previously separated chrominance component, and as shown in FIG. 3, the chrominance component may be in a state in which chrominance for a non-skin region is removed by a skin-pixel filter and only chrominance for a skin region remains.

FIG. 7 is a flowchart showing one example of a method for estimating a heart rate based on the corrected image, according to one embodiment of the present invention.

Since FIG. 7 may be implemented by the heart rate estimation device 100 described in FIGS. 1 and 2, a description will be made with reference to FIGS. 1 and 2, and hereinafter, a description overlapping the above-described contents will be omitted.

The serial image acquisition unit 210 acquires a serial image (S710).

The region of interest extraction unit 220 extracts a region of interest from the acquired serial image (S720).

The color space conversion unit 230 converts a color space of the extracted region of interest from an RGB region to a YCbCr region (S730).

The weighted value calculation unit 240 calculates a weighted value applied to the serial image by inputting the conversion result into a learning model (S740).

The corrected image generation unit 250 generates a serial image in which the region of interest is corrected by applying the calculated weighted value to the serial image (S750).

The heart rate estimation unit 260 estimates a heart rate of a person included in the corrected serial image by analyzing the region of interest of the corrected serial image (S760).

According to the present invention, a low-illumination effect may be improved by amplifying illumination of a low-illumination environment image.

In addition, according to the present invention, a more accurate heart rate waveform may be obtained by estimating a heart rate of the rPPG method using an image with an improved low-illumination effect.

The embodiment according to the present invention described above may be implemented in the form of a computer program that may be executed on a computer through various components, and the computer program may be recorded in a computer-readable medium. Examples of the computer-readable medium may include magnetic media such as hard disks, floppy disks, and magnetic tape; optical media such as CD ROMs and DVDs; magneto-optical media such as floptical disks; and hardware devices that are specially configured to store and execute program instructions, such as ROM, RAM, flash memory, and the like.

Meanwhile, the computer program may be specially designed and configured for the present invention or may be known to and usable by those skilled in the field of computer software. Examples of the computer program may include both machine code, such as produced by a compiler, and files containing higher level code that may be executed by the computer using an interpreter.

The specific implementations described in the present invention merely are embodiments and are not intended to limit the scope of the present invention in any case. For clarity of the specification, the description of conventional electronic components, control systems, software, and other functional aspects of the above systems may be omitted. In addition, the connections or connecting members of lines between the components shown in the drawings exemplify functional connections and/or physical or circuit-wise connections, and alternative or additional various functional connections, physical connections, or circuit-wise connections may be embodied in actual devices. In addition, unless specifically stated as “essential”, “important”, or the like, the corresponding component may not be required for application of the present invention.

The use of the term “the” or a similar directional term in the specification (in particular, in claims) of the present invention may correspond to both the singular and the plural. In addition, when a range is disclosed in the inventive concept, inventions to which individual values belonging to the range are applied are included (if there is no disclosure opposed to this), and this is the same as if each of the individual values forming the range is disclosed in the detailed description of the present invention. Finally, for steps forming the methods according to the present invention, if an order is not clearly disclosed or, if there is no disclosure opposed to the clear order, the steps can be performed in any order deemed proper. The present invention is not necessarily limited to the disclosed order of the steps. The use of all illustrations or illustrative terms (for example, such as, etc.) in the present invention is simply to describe the present invention in detail, and the scope of the present invention is not limited due to the illustrations or illustrative terms unless they are limited by claims. In addition, it will be understood by those of ordinary skill in the art that various modifications, combinations, and changes can be formed according to design conditions and factors within the scope of the attached claims or the equivalents.

1. A method for estimating a heart rate based on a corrected image, the method comprising:
acquiring a serial image;
extracting a region of interest (ROI) from the acquired serial image;
converting a color space of the extracted region of interest from an RGB region to a YCbCr region;
calculating a weighted value applied to the serial image by inputting a conversion result into a learning model;
generating a serial image in which the region of interest is corrected by applying the calculated weighted value to the serial image; and
estimating a heart rate of a person included in the corrected serial image by analyzing the region of interest of the corrected serial image.

2. The method of claim 1, wherein the acquiring of the serial image includes acquiring a serial image of 30 fps or greater using an RGB camera,
the serial image includes a facial region of the person, and
the region of interest is the facial region of the person.

3. The method of claim 1, wherein the serial image includes a low-illumination environment image and a general environment image, and
the calculating of the weighted value is performed by dividing the conversion result into a chrominance component and a luminance component, inputting the luminance component into a Retinex-model, and decomposing each of the low-illumination environment image and the general environment image into an illumination component and a reflectance component to perform learning.

4. The method of claim 3, wherein the generating of the corrected image includes an adjustment step of increasing the illumination component and reducing noise of the reflectance component by applying the calculated weighted value to the low-illumination environment image, and a step of recomposing adjusted illumination component and reflectance component with the chrominance component.

5. A device for estimating a heart rate based on a corrected image, the device comprising:
a serial image acquisition unit that acquires a serial image;
a region of interest (ROI) extraction unit that extracts a region of interest from the acquired serial image;
a color space conversion unit that converts a color space of the extracted region of interest from an RGB region to a YCbCr region;
a weighted value calculation unit that calculates a weighted value applied to the serial image by inputting a conversion result into a learning model;
a corrected image generation unit that generates a serial image in which the region of interest is corrected by applying the calculated weighted value to the serial image; and
a heart rate estimation unit that estimates a heart rate of a person included in the corrected serial image by analyzing the region of interest of the corrected serial image.
